(19) United States
(12) Patent Application Publication
(10) Pub. No.: US 2018/0021660 A1
(43) Pub. Date: Jan. 25, 2018

(54) Oral Devices and Methods of Making

(71) Applicant: Americo Fernandes, Winnipeg (CA)

(72) Inventor: Americo Fernandes, Winnipeg (CA)

(21) Appl. No.: 15/655,687

(22) Filed: Jul. 20, 2017

Related U.S. Application Data
(60) Provisional application No. 62/365,527, filed on Jul. 22, 2016.
(60) Provisional application No. 62/451,355, filed on Jan. 27, 2017.
(60) Provisional application No. 62/512,261, filed on May 30, 2017.

(51) Int. Cl. A63B 71/08; A61C 9/00; A61M 16/00; A61F 5/56; A61M 16/04

(52) U.S. Cl. CPC A63B 71/085; A61F 5/566; A61M 16/0488; A61M 16/0057; A61C 9/0006; A61F 2005/563; A63B 2071/086; A61M 2207/00

(57) ABSTRACT

Apparatuses for forming oral protective devices and airway management devices (e.g., to discourage snoring) are described, as well as methods of using such apparatuses for making oral protective devices and airway management devices, and devices made according to such methods and with such apparatuses. The apparatuses can comprise arch-shaped elements with any of several features that allow ease of use, ease of customization to individual users, and implementation of airways desirable for applications such as airway management.

### CROSS-REFERENCE TO RELATED APPLICATIONS

This application claims priority as a non-provisional of U.S. provisional application 62/512,261 filed May 30, 2017; 62/451,355 filed Jan. 27, 2017; and 62/365,527 filed Jul. 22, 2016, each of which is incorporated by reference herein.

### BACKGROUND

Oral protective devices, sometimes called OPDs or mouth guards, are devices worn over or in contact with teeth. OPDs can be used to protect teeth from injury, for example from teeth grinding and from injuries during sports or other physical activity. OPDs are recommended for anyone who plays contact sports such as football, boxing, soccer, ice hockey, basketball, lacrosse, and field hockey. Even those participating in noncontact sports or other activities, e.g. skateboarding, mountain biking, gymnastics) that might pose a risk of injury to the mouth can benefit from wearing an OPD.

Currently, three types of OPDs are available: stock, boil and bite, and custom-fitted.

Stock OPDs are preformed and come ready to wear. They are inexpensive and can be bought at most sporting good stores and department stores. However, their fit is not readily adjustable, they are bulky, they make breathing and talking difficult, and they provide little or no protection. Dentists do not usually recommend their use.

Boil and bite OPDs also can be bought at many sporting goods stores and can offer a better fit than stock OPDs. A boil and bite OPD is made from thermoplastic material. It is placed in hot water to soften, then placed in the mouth and shaped around the teeth using finger and tongue pressure.

Custom-fitted OPDs are individually designed and made in a dental office or a professional laboratory based on a dentist's instructions. First, the dentist makes an impression of the teeth. An OPD is then molded over the model using a special material. Due to the use of the special material and because of the extra time and work involved, this custom-made OPD is more expensive than the other types, but it provides the most comfort and protection.

Generally, OPDs cover the upper teeth only, but in some instances (such as the presence of braces or another fixed dental appliance on the lower jaw), a dentist will make an OPD for the lower teeth as well. An effective OPD should be comfortable, resist tears, be durable and easy to clean, and should not restrict breathing or speech.

An OPD used to prevent tooth damage from tooth grinding at night is often called a nocturnal bite plate or bite splint.

There is a need for inexpensive OPDs that provide better fit, for comfort and for more effective protection. Such OPD technology would be even more valuable if it were able to manage a wearer's airway during sleep, offering relief from snoring.

### SUMMARY OF THE INVENTION

Example embodiments provide an airway management device, comprising a material compatible with a human mouth shaped to conform to the arch of a user's teeth and to at least one of the user's upper or lower teeth, and having one or more air passages extending from a lingual side of the device when held between the user's teeth to the buccal side. The material can comprise silicone. The device can have a cross-section that is U-shaped, with at least a portion of the interior of the U-shaped cross-section conforming to at least a portion of the user's upper or lower teeth. The device can have a cross-section that is H-shaped, with at least a portion of the interior of an upper portion of the H-shaped cross-section conforming to at least a portion of the user's upper teeth, and at least a portion of the interior of a lower portion of the H-shaped cross-section conforming to at least a portion of the user's lower teeth. The air passage can be configured to connect with a CPAP machine.

Example embodiments provide a method of producing an airway management device, comprising (a) providing a tray configured to match an arch of a user's teeth, where the tray has a lingual face and a buccal face that together define an internal channel shaped to accommodate at least a portion of the user's upper teeth, lower teeth, or both; and wherein the tray defines one or more airway channels extending from the lingual face to the buccal face; (b) applying settable material to the internal channel but not in the one or more airway channels; (c) causing the user to bite on the tray, forcing one or more of the user's teeth into the internal channel and encouraging the settable material to conform to the shape of the one or more of the user's teeth; (d) waiting for the settable material to set.

Example embodiments provide a method of producing an airway management device, comprising: (a) providing a buccal shield configured to match an arch of a user's teeth, and wherein the buccal shield has one or more protrusions extending from the concave side of the shield a first distance; (b) applying an amount of a settable material to the concave side of the shield, surrounding the one or more protrusions, and extending less than the first distance from the concave side of the shield; (c) placing the buccal shield such that the settable material is between the user's teeth, and causing the user to bite on the settable material, forcing one or more of the user's teeth into the settable material and encouraging the settable material to conform to the shape of the one or more of the user's teeth; (d) waiting for the settable material to set; (e) removing the buccal shield and the one or more protrusions from the settable material.

Example embodiments provide a dental tray for forming dental appliances, comprising an arch element defining an arch that matches an arch of a user's teeth and having a H-shaped cross-section; wherein the arch element defines one or more air passages extending from the concave side of the arch to the convex side of the arch separated from the internal channels formed by the H-shaped cross-section. The arch element can comprise a flexible element and a bendable element that retains its shape once bent, mounted together such that the flexible element takes a shape defined by the shape to which the bendable element is bent. The bendable element can be removeable from the flexible element. The arch element can define one or more retention channels through the middle bar of the H-shaped cross-section connecting the internal channels formed by the H-shaped cross-section. The arch element can comprise a lingual flange, a buccal flange, and one or more connectors configured to mount with the lingual flange and the buccal flange and constrain the lingual flange and buccal flange to a shape that accommodates at least a portion of a user's upper or lower teeth between the lingual flange and the buccal flange. The one or more connectors can be configured to snap into openings in the lingual flange and in the buccal flange.

Example embodiments provide a kit for making a dental device, comprising a tray as described above and a quantity of settable material suitable for application into the tray and conformance with one or more teeth of a user after setting.

### BRIEF DESCRIPTION OF THE DRAWINGS

FIG. 1 is an illustration of a user of an airway management device according to the present invention.

FIG. 2 is an illustration of an airway management device according to the present invention.

FIG. 3 is an illustration of a buccal shield and airway dowels according to the present invention.

FIG. 4 is an illustration of a buccal shield and airway dowels according to the present invention.

FIG. 5 is an illustration of a buccal shield and airway dowels according to the present invention, with molding material after conformance to the shield and the dowels.

FIG. 6 is an illustration of a buccal shield and airway dowels according to the present invention, with molding material after impression by a user's bite.

FIG. 7 is an illustration of molding material prepared for use with a buccal shield and airway dowels according to the present invention.

FIG. 8 is an illustration of a buccal shield and airway dowels, disassembled, according to the present invention.

FIG. 9 is an illustration of a buccal shield and airway dowels, assembled, according to the present invention.

FIG. 10 is an illustration of a connection of a device according to the present invention to a CPAP system.

FIG. 11 is an illustration of a connection of a device according to the present invention to a CPAP system.

FIG. 12 is a schematic illustration of an example tray suitable for use in the present invention.

FIG. 13 is a schematic illustration of a cross-section of a tray as in FIG. 1.

FIG. 14 is a schematic illustration of the cross-section in FIG. 2, with silicon dental impression material placed in the tray.

FIG. 15 is a schematic illustration of the cross-section in FIG. 3, after a patient has bitten into the tray and impression material, and the impression material has set.

FIG. 16 is a schematic illustration of the cross-section in FIG. 4, after excess impression material has been trimmed away, forming a final custom OPD.

FIG. 17 is a schematic illustration of an example embodiment of a tray suitable for use in the present invention, having features that facilitate custom fit to various mouth sizes and shapes.

FIG. 18 is a schematic illustration of a top view of an example embodiment.

FIG. 19 is a schematic illustration of a side view of an example embodiment.

FIG. 20 is a schematic illustration of a lingual view of an example embodiment.

FIG. 21 is a schematic illustration of an end view of an example embodiment.

FIG. 22 is a schematic illustration of a top end view of an example embodiment.

FIG. 23 is an illustration of design considerations useful in practicing the invention.

FIG. 24 is a schematic illustration of an example embodiment, shown in section through a putty retention channel.

FIG. 25 is a schematic illustration of steps in producing a device according to an example embodiment.

FIG. 26 is a photograph of a mouthpiece produced according to the present invention.

FIG. 27 provides schematic illustrations of several suitable configurations and shapes.

FIG. 28 is a schematic illustration of components of an example embodiment, as separate pieces and after assembly into a dental tray.

FIG. 29 is a lingual view of an example embodiment of a dental tray according to the present invention.

FIG. 30 is a buccal view of an example embodiment of a dental tray according to the present invention.

FIG. 31 is a top view of an example embodiment of a dental tray according to the present invention.

FIG. 32 comprises top and lingual views of an example embodiment of a dental tray according to the present invention.

### DESCRIPTION OF INVENTION

### Example Embodiment A

Embodiments of the present invention provide devices that can be produced in a home or office setting, and that are useful in applications such as airway management for anti-snoring, and anti-grinding. Embodiments can be made from silicone, which as a soft material can enable very comfortable devices for the user. The device can open up the user's airway during sleep, by advancing the lower jaw forward, keeping the teeth slightly apart.

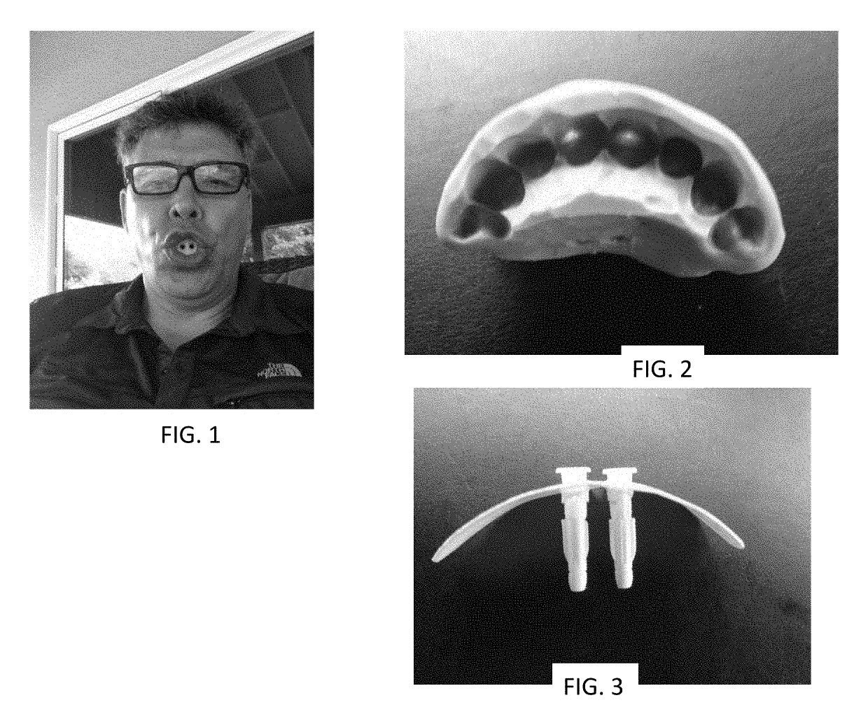

The method does not require advanced or expensive equipment, and can be practiced in a home or office setting. The user can start with a kit comprising elements not commonly available in the home. As an example, the kit can comprise a customizable plastic buccal dental impression tray with biting components designed to hold setting silicone around the teeth in specific position, between the upper and lower anterior teeth as shown in FIGS. 1-9 and biting edge to edge.

The kit utilizes a new device, illustrated in FIGS. 3-6 and 7-9, which provides a novel dental impression tray that holds the setting silicone material around the upper and lower teeth in a very specific position. The set silicone becomes the device when separated from the impression tray. The novel dental impression tray is reusable. It comprises a buccal shield that fits over the anterior faces of the upper and lower teeth, extending from the central teeth to the second premolars or further back to include the molar teeth. The shield part of the dental impression tray can be made of plastic and can also be made from other materials. It can be customizable, for example using scissors, to achieve a custom and comfortable fit between the insides of lips and the anterior faces of lower and upper teeth.

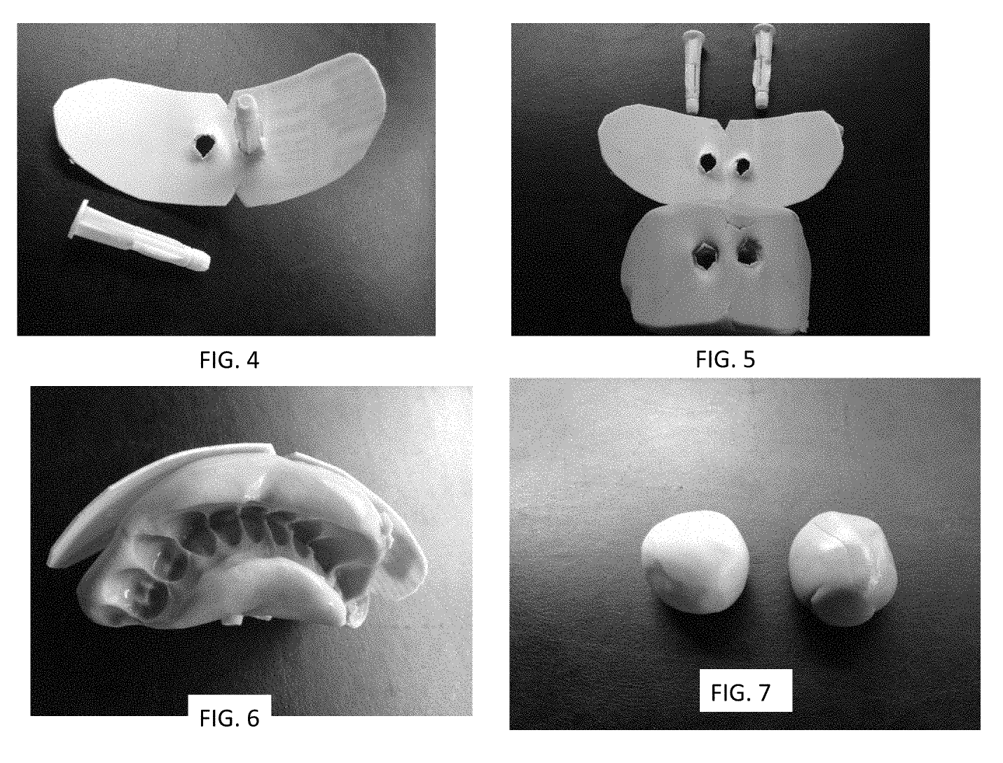

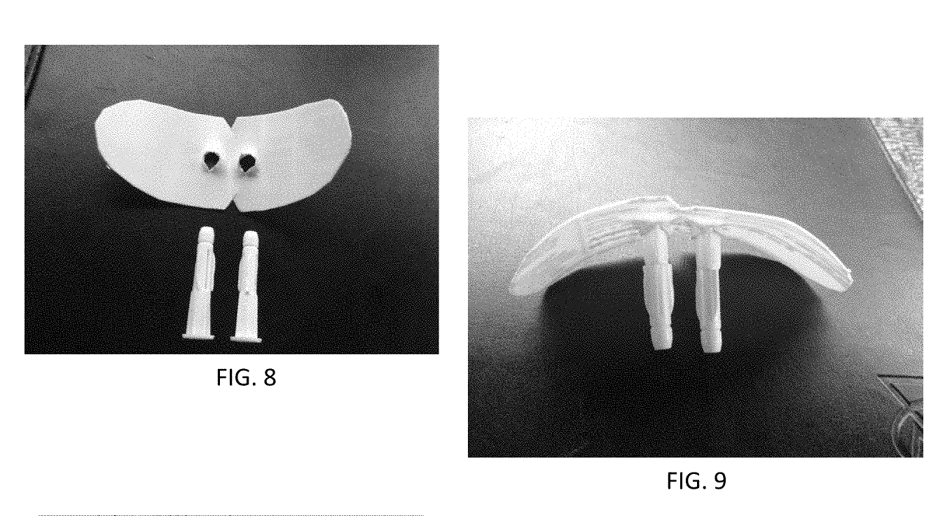

The buccal dental impression shield tray can be designed to accommodate the attachment of two or more removable or fixed circular or other shaped smooth dowels, as illustrated in FIGS. 3-4. Note the dowels can also be integral to the shield. These dowels project into the lingual, beyond the lingual walls of the teeth. These dowels can be approximately 3 to 5 mm in diameter (for some common applications, but can be more or less, depending on the application) and 10 to 15 mm in length (for some common applications, but can be more or less, depending on the application). These dowels can attach to the dental impression shield at an appropriate position as shown in the examples in the figures. In an example embodiment shown in FIGS. 3 and 8, the dowels are centered side by side horizontally and equidistant between upper and lower teeth on the dental impression shield. These dowels are engaged between the incisal edges of the anterior upper and lower teeth. The buccal dental impression tray is held correctly in place by the user when biting on the lingual projecting dowels with teeth edge to edge. The tray encourages the user to move the lower teeth forward to meet the upper teeth on opposite sides of the dowels.

To produce an exemplary device, an appropriate amount of silicone can be mixed, for example by hand kneading, and placed on the lingual aspect of the customized buccal dental impression tray, as illustrated in FIGS. 5-7. The soft unset mixed silicone is molded into place with fingers, completely engaging the lingual projecting dowels.

The user places the customized buccal dental impression tray with dowels and mixed unset silicone correctly into the mouth by biting through the unset silicone until the edges of the upper and lower anterior teeth make contact with the lingual projecting dowels. The edges of the teeth directly oppose each other when biting onto the dowels. The user holds the teeth in this position until the silicone is fully set. Once the silicone is set, the patient forces the teeth apart, releasing the set silicone and tray complex from the teeth, as illustrated in FIG. 6.

The custom buccal dental impression tray and dowels can then be separated from the set silicone outside the mouth. The set silicone complex results in a device that fits perfectly by engaging the upper and lower teeth in the desired position, as illustrated in FIGS. 1-2. The set silicone device also comprises two or more channels between the upper and lower teeth facilitating better breathing when the device is in place in the mouth. These breathing channels are made when the dowels are removed from the set silicone.

The silicone complex can be custom trimmed with scissors or blade to enhance user comfort during sleep.

The customizable buccal dental tray with the dowels (2 round dowels shown in the figures, though other shapes and numbers of dowels can be used, and other airway mechanisms can also be used such as permeable material for at least the airway portion of the device itself) can be reused to fabricate a new silicone device whenever needed. Silicone material can be ordered separately from the customized buccal dental impression tray with dowels thereby reducing the cost to the user.

A device according to the preceding description can be used to discourage snoring. Inserting into the mouth a device like that described above before sleep can facilitate a more open airway and reduce the occurrence or extent of snoring, as illustrated in FIG. 1.

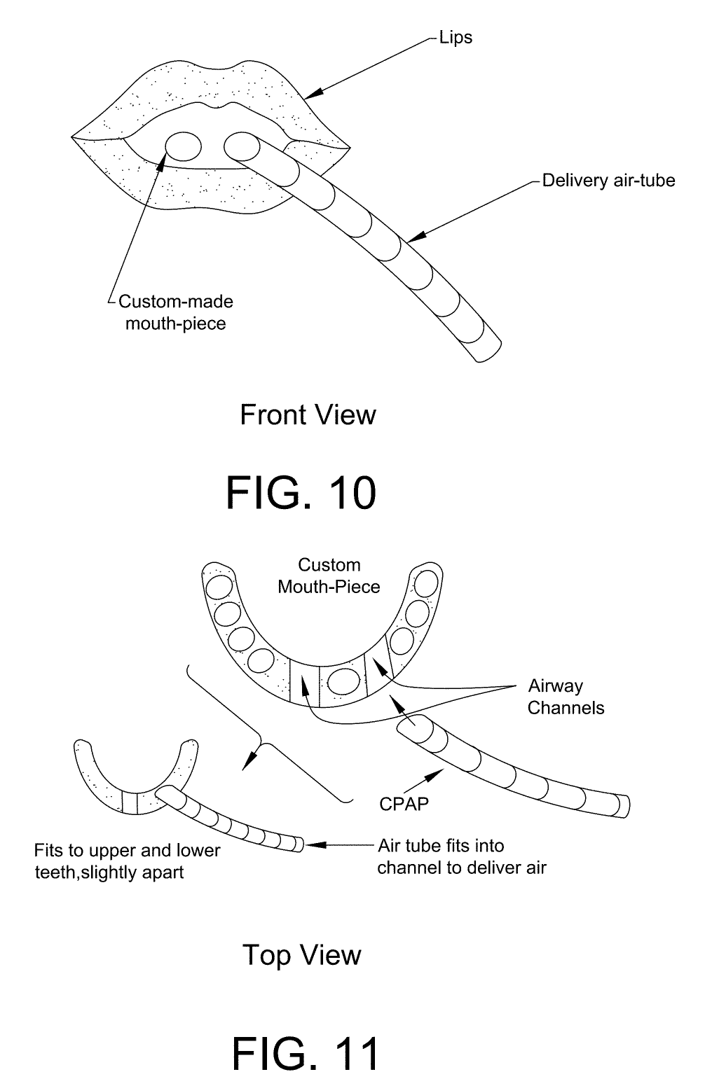

A device like that described herein can also be used in connection with a continuous positive airway pressure (CPAP) application, as illustrated in FIGS. 10-11. The CPAP air supply can be routed, for example using tubes and compatible connectors, to the openings in the device. The device encourages the airway to stay open, and the user's mouth retains the device in position. The simple, customized device can reduce the need to obtrusive masks and head straps.

### Example Embodiment B

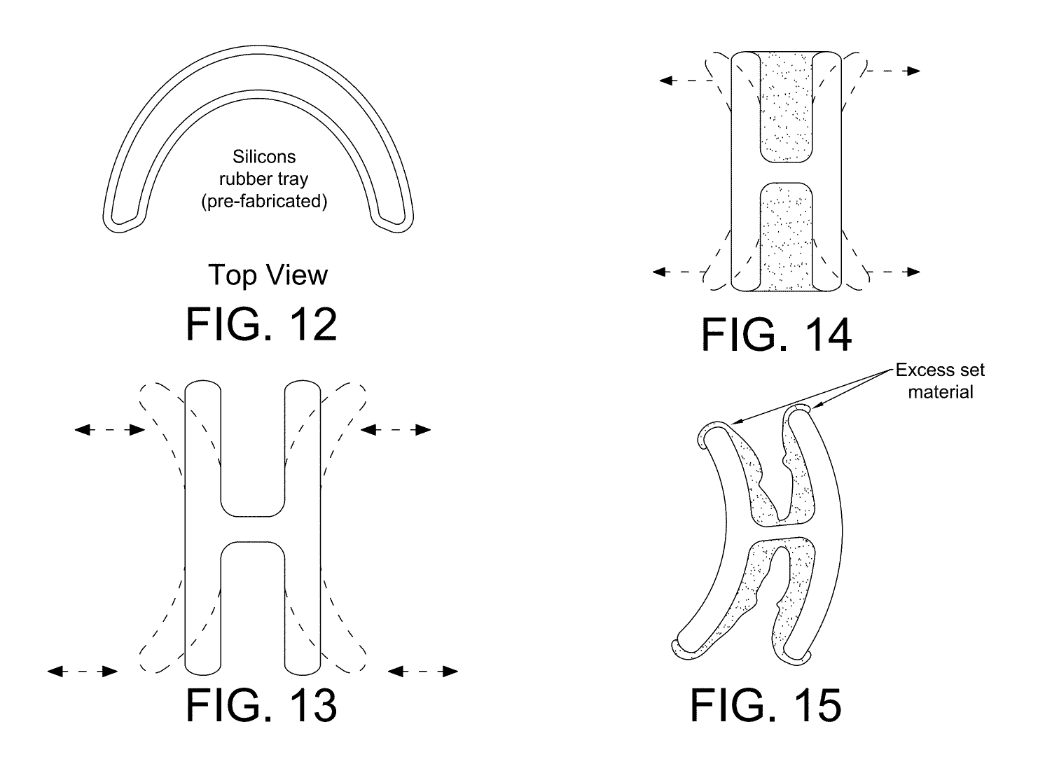

FIG. 12 is a schematic illustration of an example tray suitable for use in the present invention. The example tray is made of a material such as silicone rubber, compatible with use as an OPD and having sufficient stiffness to retain its shape in the presence of normal jaw activity and in the presence of tooth grinding, sports impacts, or other likely use conditions. Such trays can be made in various sizes and shapes to accommodate various mouth sizes and shapes. Such trays can be mass produced in a limited number of sizes and shapes, e.g., small, medium, and large, facilitating economies of scale in production and consequent low cost.

FIG. 13 is a schematic illustration of a cross-section of a tray as in FIG. 12. The cross-section shown assumes an OPD to protect both upper and lower teeth. If only upper or lower teeth protection is desired then the tray can have a cross-section like a U instead of an H as shown. The material of the tray can be sufficiently flexible to allow some deformation to better match the shape of a user's mouth. The material can also be compatible with trimming with normal household implements such as scissors or knives.

Silicone rubber can have the desired properties for such trays. The raw material of choice for American infant pacifiers is silicone, which is more expensive than latex but has superior performance. Silicones are synthetic polymers that are relatively chemically inert, stable at high temperatures, and resist oxidation. Silicone is able to survive a boil and cool cycle far better than latex and therefore superior for products requiring sterilization. Some pacifier manufacturers buy silicone that is “certified,” meaning it is of a superior grade for use. In addition, this means that silicone devices can be cleaned in a dishwasher without fear of distortion, unlike plastic based devices. Silicone oral devices do not tolerate bacterial growth, therefore they do not stink when compared to porous plastic oral devices. Accordingly, silicone can be a suitable material for both the tray and the filler material, described below.

FIG. 14 is a schematic illustration of the cross-section in FIG. 13, with unset filler material (also called impression material) placed in the tray. Suitable material is commonly available as silicone dental impression material, often as a two part mix. The material is placed in the tray, in the opening in the cross-section as shown in the figure.

FIG. 15 is a schematic illustration of the cross-section in FIG. 14, after a patient has bitten into the tray and impression material, and the impression material has set. The filler material is soft and can be displaced by mechanical force as the user bites into the tray. After placing the material in the tray, the user bites into it and forces the filler material around the teeth, with excess forced toward the edges of the opening as shown in the figure. The user maintains the mouth's position until the filler material sets, for example a few minutes with commonly available impression materials. The setting of the filler material provides a custom fit to the user's teeth, and holds the tray in the shape that matches the user's mouth.

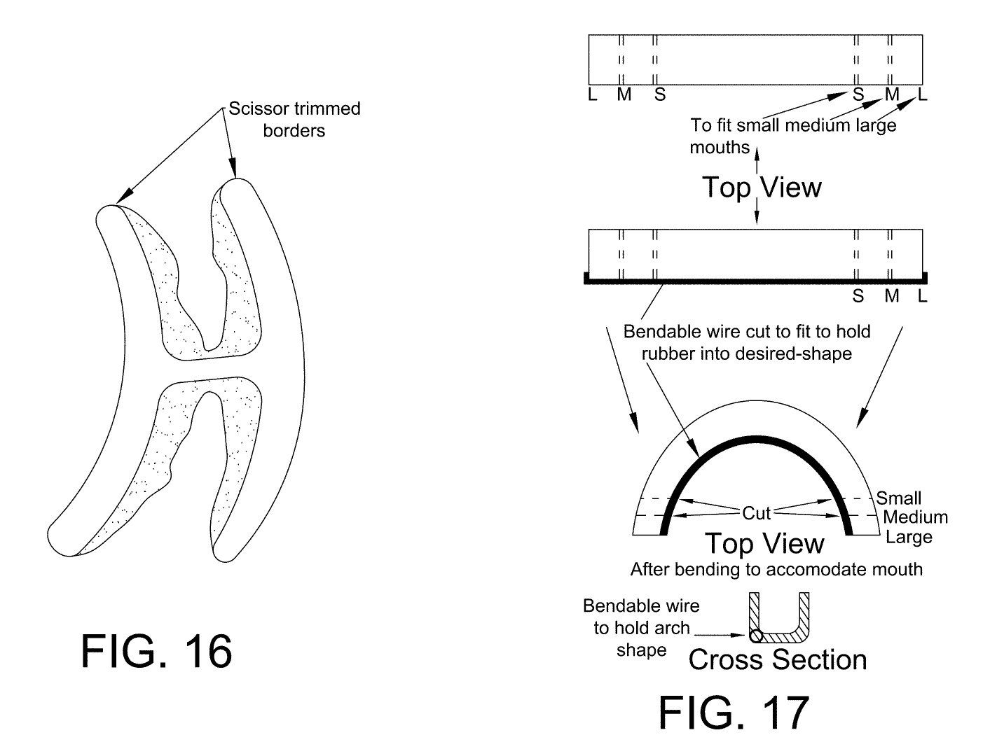

FIG. 16 is a schematic illustration of the cross-section in FIG. 15, after excess impression material has been trimmed away, forming a final custom OPD. Excess filler material, and any excess tray material, can be trimmed with household tools such as scissors or knives. Any areas of the tray or filler material that cause irritation can similarly be cut away. The resulting OPD has the relatively rigid protective shape imparted by the tray, and a perfect interior fit to the user's mouth and teeth since the interior shape of the OPD was formed by impression of the user's actual mouth and teeth.

FIG. 17 is a schematic illustration of an example embodiment of a tray suitable for use in the present invention, having features that facilitate custom fit to various mouth sizes and shapes. The example in the figure is for a one-sided OPD (either top or bottom teeth), and accordingly has a U cross-section. An H cross-section as in the previous example can also be used if a two-sided OPD is desired.

The tray begins as an element with a U-shaped cross-section, with a curve that is compatible with typical dental arch curvatures. The element is of a material that can be cut to various lengths, and that is flexible enough to be bent to conform to the exact arch shape of the user. A user can position the tray in the mouth, and trim the ends of the element until the tray fits the overall length of the user's arch, and bend the tray until it matches the shape of the user's arch. Setting of the filler material can be sufficient to hold the tray in the desired arch shape.

The element can also comprise a bendable component, inserted into tray or formed as part of the tray. The bendable component can comprise, as an example, a bendable metal wire or ribbon. The bendable component can mount in various locations relative to the tray. As shown in the figure, it can mount near the bottom of the tray, on the side that will be inside the teeth when the OPD is in use. Such positioning can help prevent the bendable element from contributing to injuries from impacts. The bendable element can be flexible enough to allow the user to easily conform the tray (including the bendable element) to the user's exact arch shape, while stiff enough to hold the tray in that shape while the filler material is inserted, the user bite achieved, and the filler material allowed to set. The bendable element can remain part of the OPD. Alternatively, the bendable element can be removed from the OPD once the filler material has set, provided the filler material provides sufficient stiffness to keep the OPD in the desired arch shape. As an example, the bendable element can comprise a metal ribbon on the outer surface of the tray, which can be peeled off once the filler material has set.

As another example embodiment, the tray can be configured such that the interior volume of the tray corresponds to the desired final volume of the OPD. The filler material can be introduced to the tray and an impression made by the user's teeth as described before. After the filler material has set, the tray can be separated from the filler material and the filler material used as the OPD. This can be more difficult for the user, since the user's mouth must accommodate a larger tray while the impression is being made. It can provide more control over the final OPD, for example by using a filler material that, when set, is more flexible than would be suitable for a tray for making impressions. The more flexible filler material, and hence more flexible OPD, can provide for more comfort or lower external visibility when wearing.

As another example embodiment, a tray with an H cross-section can be supplied, with the cross-section configured such that the lower jaw is advanced slightly relative to the upper jaw. The resulting OPD can also provide benefit as an anti-snoring device, since the advanced lower jaw position can help open the user's airway and reduce snoring. Passages for direct airways through the OPD can be provided, for example by openings in the tray or removable dowels as described in connection with other example embodiments.

When making OPDs for users with dental appliances, such as braces, already in the mouth, the appliance can be masked prior to making the impression. As an example, wax can be used to cover the braces before making the impression. The filler material will thus not get caught in the braces, but will still tightly conform to the shape of the teeth.

OPDs intended for protection during sports activities can be made using a U shaped tray impressed by the upper jaw. OPDs intended for night guard, or anti-grinding, applications, can be made using a U shaped tray impressed by the lower jaw. H shaped trays can be used for either application, and can be especially useful in anti-snoring applications so that the upper and lower jaws can be properly positioned.

### Example Embodiment C

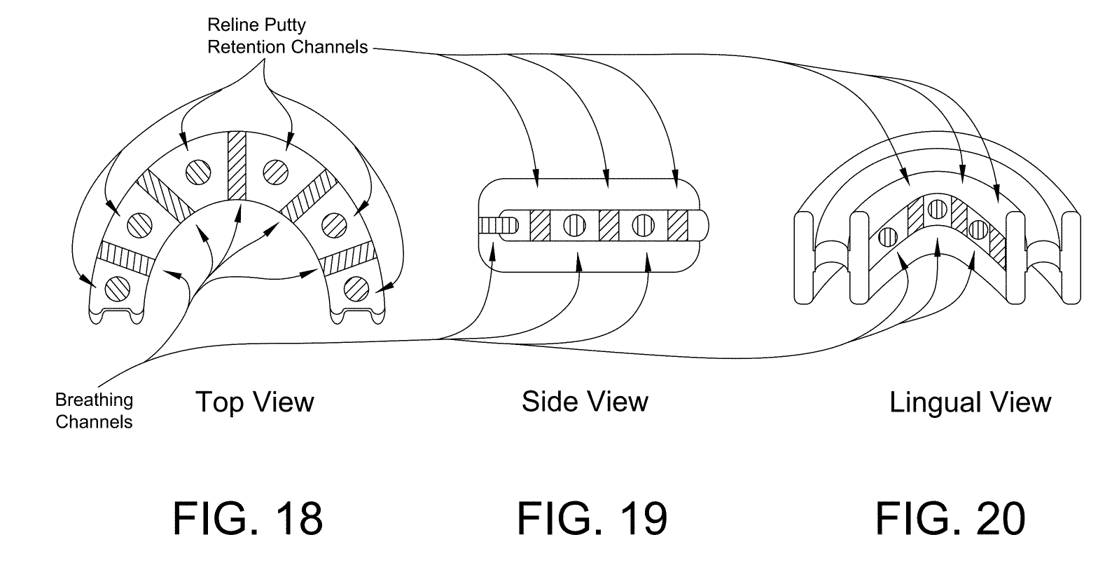

The example embodiment comprises a tray, for example of a material such as plastic or semi-rigid cured silicone or rubber. The tray comprises a central web that separates the user's upper teeth from the user's lower teeth, as illustrated in FIG. 18. Mounted with the central web are inner and outer walls, that, together with the central web, form an upper channel with space for the user's upper teeth and a lower channel with space for the user's lower teeth. The tray is shaped to fit around a user's teeth, as described in connection with other example embodiments. The tray has one or more breathing channels that form open passages through the inner wall, the central web, and the outer wall, as illustrated in FIGS. 19-20. The breathing channels do not communicate with the channels corresponding to the user's teeth, so that formable material (e.g., putty) placed in the channel does not compromise the air flow characteristics of the breathing channels. The tray further defines one or more retention channels, extending through the central web and allowing communication between the upper and lower channels, as illustrated in FIG. 18-20. Formable material in the upper channel accordingly communicates with formable material in the lower channel, and such communication can discourage the formed (and cured, where applicable) material in either channel from peeling or falling out of the corresponding channel.

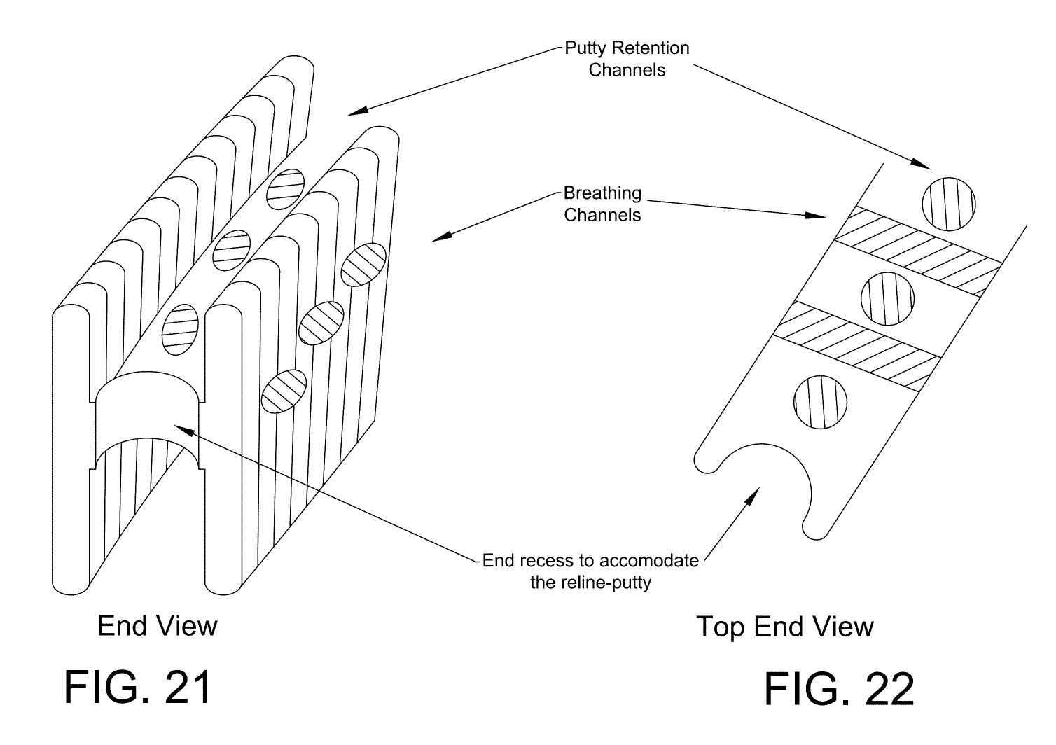

The central web can have ends with recesses as illustrated in FIG. 21. Putty from the upper and lower channels can communicate around the end of the central web in the space allowed by the recess. This can facilitate retention of the putty in the channels, and avoid irritation from hard or sharp edges at the end of the web that might otherwise contact the tissues of the user.

The tray can be used by dentists with their preferred silicone putty to fabricate custom anti-grinding and anti-snoring mouthpieces. The mouthpieces can be made in the office, without requiring return visits or external fabrication facilities.

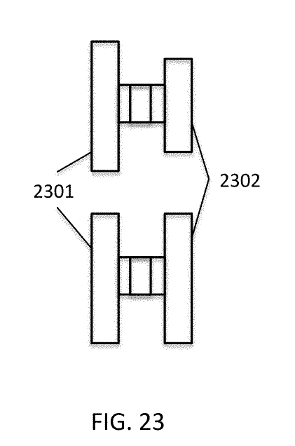

FIG. 23 presents illustrations relating to design considerations useful in practicing the invention. The facial flange thickness can be selected so that the facial flange 2301 is of similar thickness as the lingual flange 2302 thickness, providing advantageous performance. The facial flange height can be consistent, as in FIG. 23. The facial flange 2301 height and lingual flange 2302 height can be similar, as in FIG. 23, providing advantageous performance.

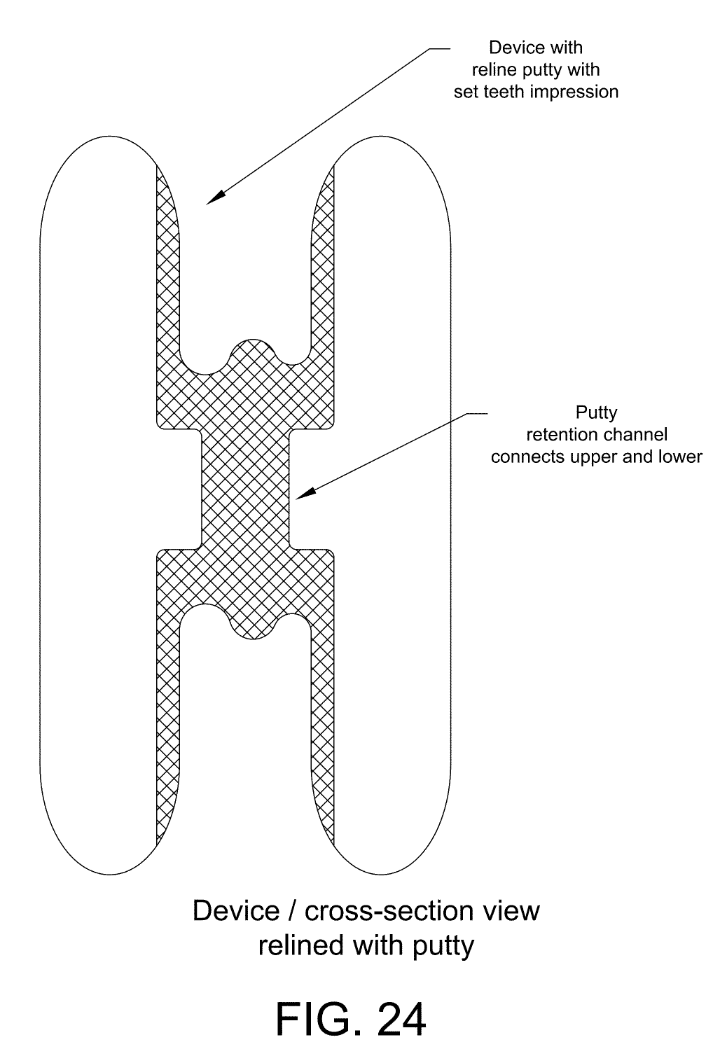

FIG. 24 is a schematic illustration of an example embodiment, shown in section through a putty retention channel. A tray 2401 like that described above has been loaded with putty 2402. The section exposes one putty retention channel 2403, showing how putty in the retention channel can help retain the putty in the upper and lower channels. Retention can also be encouraged with depressions or protrusions that engage the putty and mechanically oppose motion of the putty out of the tray. FIG. 27 provides schematic illustrations of several suitable configurations and shapes. As an example, recesses can be sized so that they do not penetrate the inner or outer wall; 1 to 1.5 mm can be suitable in some applications. As an example, protrusions can be sized so that they do not interfere with the teeth as the user is biting to make the impression; 1 to 1.5 mm can be suitable in some applications.

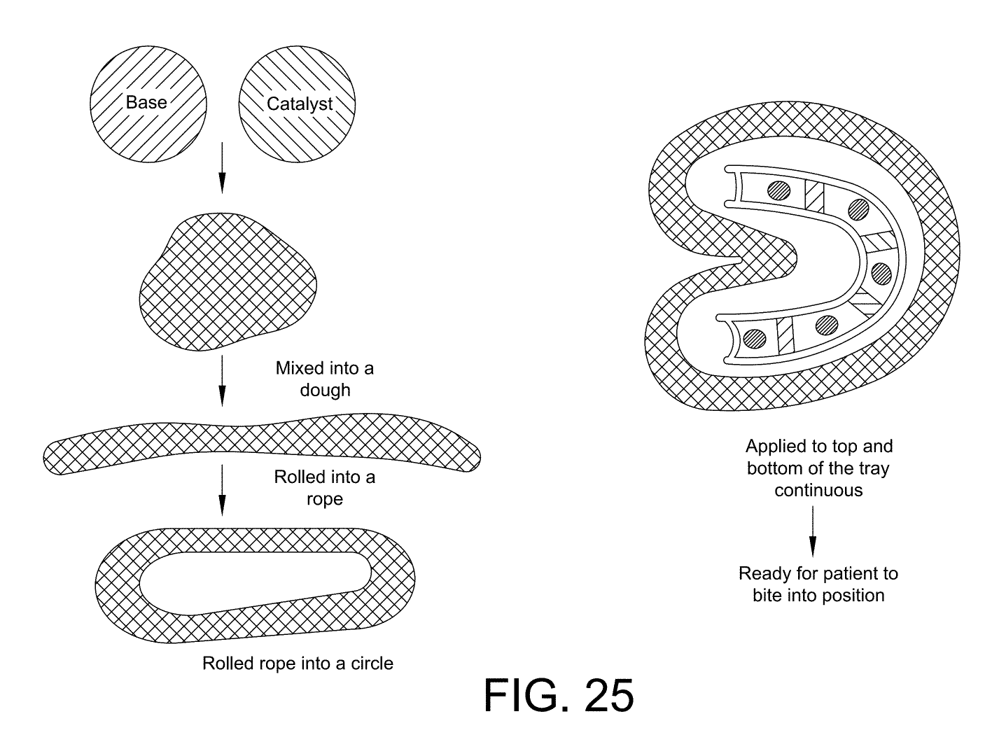

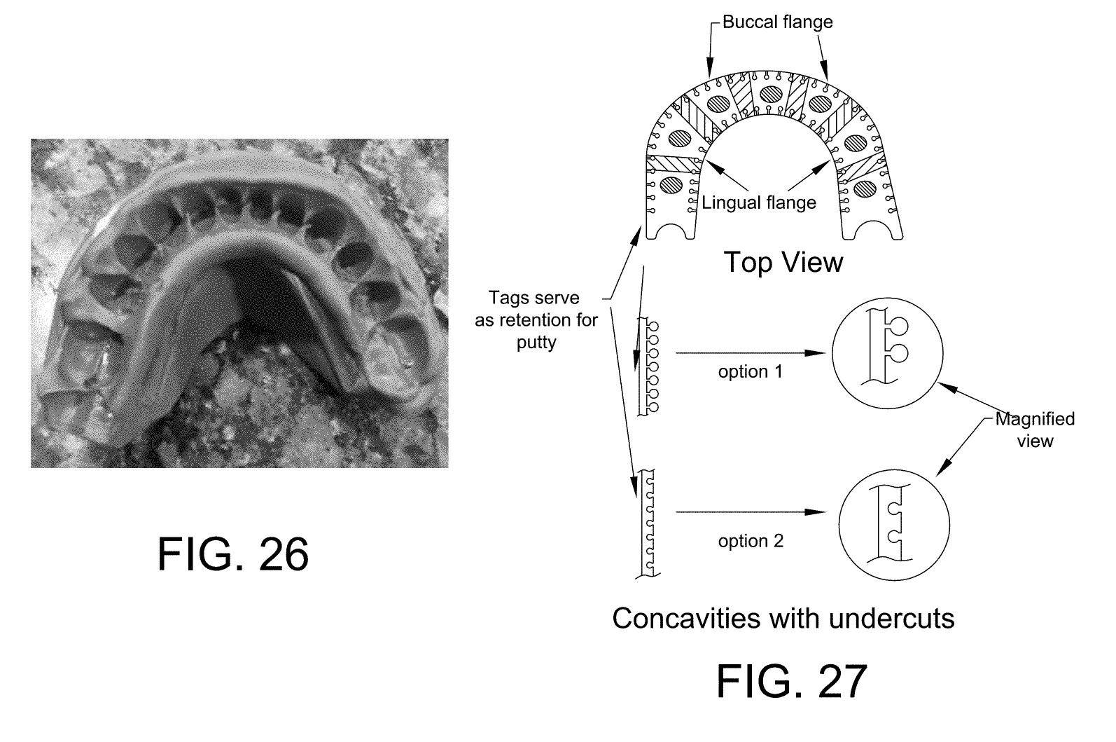

FIG. 25 is a schematic illustration of steps in producing a device according to an example embodiment. A base and catalyst are mixed, according to materials and methods known to those skilled in the art, such as materials and methods used by dentists to make dental impressions. The resulting dough is rolled into a rope shape, whose ends are matched to form a circle. The circle can be inserted into the upper channel, and then wrapped around the recessed ends and inserted into the lower channel. The tray is then ready for a user to bite, forming a match of the resulting cured putty to the actual shape of the user's teeth. FIG. 26 is a photograph of a mouthpiece produced according to the present invention.

### Example Embodiment D

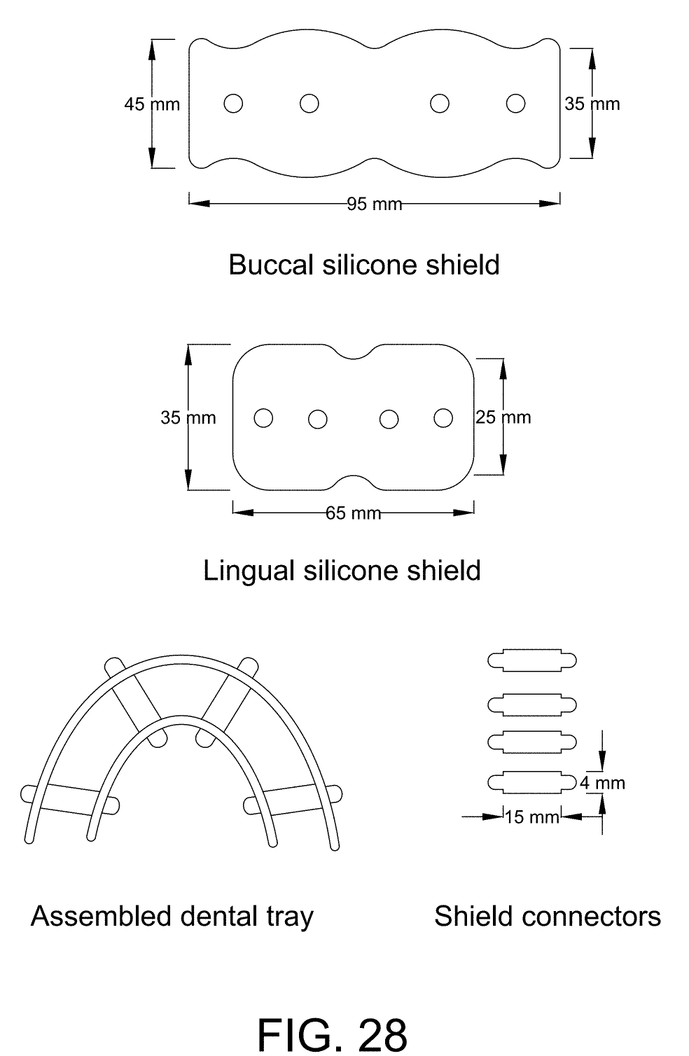

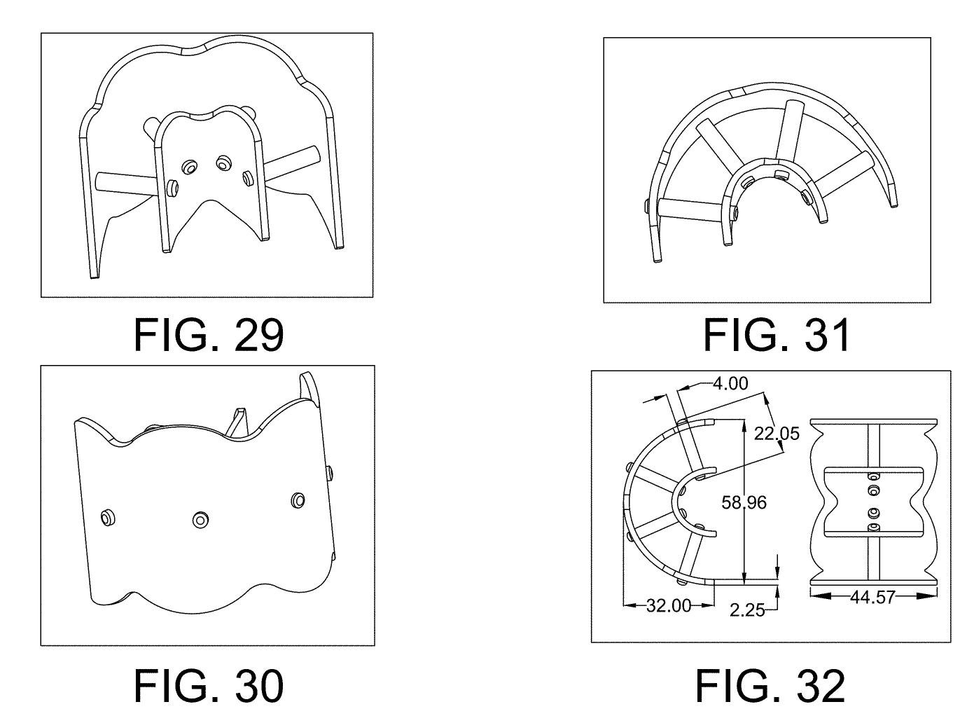

FIGS. 28, 29, 30, 31, and 32 are illustrations of an example embodiment of the present invention. This example embodiment provides a dual arch tray system that can be separated and removed from the already set silicone once the patient has bitten into it. The system can comprise a buccal shield, e.g., of silicon; a lingual shield, e.g., of silicon, and a plurality of shield connectors, that can be assembled into a dental tray. The example embodiment provides a snap-on tray that can be easily separated from the set silicone anti-snore guard. The tray can be considered as analogous to a concrete form, which holds the cement in place until it sets and then is removed leaving the set desirable shape system in place. As an example, the buccal and lingual shields can be made out of standard rubber currently used for dental whitening trays. The connectors can be made from plastic, as an example, and can be configured to snap on to buccal and lingual shields that form the dual arch tray. These connectors can be about 4 mm in diameter, which corresponds to a suitable amount of separation between the upper and lower teeth when creating an anti-snore guard. The intra-distance between the shields can be 15 mm. The drawings show 4 connectors per tray for demonstration purpose only. Other numbers of connectors, for example 5 or 6, can also be suitable. A single connector, for example a connector with multiple attachment points to each flange, can also be used. The example embodiment can easily be reused on the same patient until a desired silicone device is achieved.

Although the present invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

We claim:

1. An airway management device, comprising a material compatible with a human mouth shaped to conform to the arch of a user's teeth and to at least one of the user's upper or lower teeth, and having one or more air passages extending from a lingual side of the device when held between the user's teeth to the buccal side.

2. An airway management device as in claim 1, wherein the material comprises silicone.

3. An airway management device as in claim 1, wherein the device has a cross-section that is U-shaped, with at least a portion of the interior of the U-shaped cross-section conforming to at least a portion of the user's upper or lower teeth.

4. An airway management device as in claim 1, wherein the device has a cross-section that is H-shaped, with at least a portion of the interior of an upper portion of the H-shaped cross-section conforming to at least a portion of the user's upper teeth, and at least a portion of the interior of a lower portion of the H-shaped cross-section conforming to at least a portion of the user's lower teeth.

5. An airway management device as in claim 1, wherein an air passage is configured to connect with a CPAP machine.

6. A method of producing an airway management device as in claim 15, comprising:
(a) providing a tray configured to match an arch of a user's teeth, where the tray has a lingual face and a buccal face that together define an internal channel shaped to accommodate at least a portion of the user's upper teeth, lower teeth, or both; and wherein the tray defines one or more airway channels extending from the lingual face to the buccal face;
(b) applying settable material to the internal channel but not in the one or more airway channels;
(c) causing the user to bite on the tray, forcing one or more of the user's teeth into the internal channel and encouraging the settable material to conform to the shape of the one or more of the user's teeth;
(d) waiting for the settable material to set.

7. A method of producing an airway management device as in claim 15, comprising:
(a) providing a buccal shield configured to match an arch of a user's teeth, and wherein the buccal shield has one or more protrusions extending from the concave side of the shield a first distance;
(b) applying an amount of a settable material to the concave side of the shield, surrounding the one or more protrusions, and extending less than the first distance from the concave side of the shield;
(c) placing the buccal shield such that the settable material is between the user's teeth, and causing the user to bite on the settable material, forcing one or more of the user's teeth into the settable material and encouraging the settable material to conform to the shape of the one or more of the user's teeth;
(d) waiting for the settable material to set;
(e) removing the buccal shield and the one or more protrusions from the settable material.

8. A dental tray for forming dental appliances, comprising an arch element defining an arch that matches an arch of a user's teeth and having a H-shaped cross-section; wherein the arch element defines one or more air passages extending from the concave side of the arch to the convex side of the arch separated from the internal channels formed by the H-shaped cross-section.

9. A tray as in claim 8, wherein the arch element comprises a flexible element and a bendable element that retains its shape once bent, mounted together such that the flexible element takes a shape defined by the shape to which the bendable element is bent.

10. A tray as in claim 9, wherein the bendable element is mounted in a removeable relationship with the flexible element.

11. A tray as in claim 9, wherein the arch element defines one or more retention channels through the middle bar of the H-shaped cross-section connecting the internal channels formed by the H-shaped cross-section.

12. A tray as in claim 9, wherein the arch element comprises a lingual flange, a buccal flange, and one or more connectors configured to mount with the lingual flange and the buccal flange and constrain the lingual flange and buccal flange to a shape that accommodates at least a portion of a user's upper or lower teeth between the lingual flange and the buccal flange.

13. A tray as in claim 13, wherein the one or more connectors are configured to snap into openings in the lingual flange and in the buccal flange.

14. A tray as in claim 9, further comprising a quantity of settable material suitable for application into the tray and conformance with one or more teeth of a user after setting.

15. A method of producing an airway management device, comprising providing a form that assists a settable material in conforming to at least a portion of a user's teeth, wherein the form includes one or more elements that provide airways through the settable material.
